(19) United States
(12) Patent Application Publication
(10) Pub. No.: US 2020/0197257 A1
(43) Pub. Date: Jun. 25, 2020

(54) TREATMENTS FOR DIABETIC AND PERIPHERAL NEUROPATHY

(71) Applicant: Matthew M. DiDuro, Marietta, GA (US)

(72) Inventor: Matthew M. DiDuro, Marietta, GA (US)

(21) Appl. No.: 16/226,858

(22) Filed: Dec. 20, 2018

(51) Int. Cl. A61H 23/00; A61P 25/02; A61B 18/26; A61H 23/02; A61H 23/04

(52) U.S. Cl. CPC A61H 23/008; A61P 25/02; A61N 2007/0021; A61H 23/0245; A61H 23/04; A61B 18/26

(57) ABSTRACT

A method of treating a patient with diabetic and peripheral neuropathy symptoms has the steps of establishing the severity of neuropathy symptoms and treating the patient with a combination of acoustic shock waves and light therapy.

### TECHNICAL FIELD

The present invention relates to the field of treating peripheral neuropathy and diabetic patients with acoustic pressure pulse shock waves and light therapy generally. More specifically to treating the abnormally high conditions found in a diabetic using shock waves that are generated as either focused waves at high or low energy levels or non-focused waves at preferably low energy levels or radial waves or a combination of such waves to lower the conditions to allow light therapy treatments to be effective to eradicate the neuropathy symptoms.

### BACKGROUND OF THE INVENTION

On Dec. 20, 2006 the United Nations General Assembly passed a landmark resolution recognizing diabetes as a global pandemic. This is a first for a non-infectious disease. This resolution led by the International Diabetes Federation has brought global attention to a disease that affects 246 million people living with diabetes. On Nov. 14, 2007 the UN will observe the First World Diabetes Day.

The financial burden of diabetes is tremendous. The direct and indirect costs associated with both forms of diabetes, type 1 and type 2, in the United States during 2002 were estimated to be 132 billion. The average annual health care costs for a person with diabetes are 13,243, which is 2.4 times greater than those for an individual without diabetes. In 2002, 11 percent of national health care expenditures were directed to diabetes care. The costs of treating the complications of diabetes, which both forms of the disease share in common, account for much of the health care costs associated with the disease. Although estimates of the rates of diabetes have increased since 2002, the associated cost estimates have not yet been revised; hence, the economic data given here are conservative. Clearly, the economic and societal burden of diabetes has a profound impact on the Nation.

Type 1 diabetes is an autoimmune disease in which the body's own immune system attacks and destroys specialized cells of the vascular system called beta cells. Beta cells are found within tiny clusters called islets and produce the hormone insulin. Insulin is required for survival; it sends signals to the body's cells and extremities, telling them to absorb glucose to use as a fuel. Without this vital hormone, the cells and extremities do not absorb glucose and patients can starve to death, despite having high levels of glucose in their bloodstream. An interplay of genetic and environmental factors is responsible for the onset of type 1 diabetes (as well as type 2 diabetes). Having a family member with the disease puts one at higher risk for developing type 1 diabetes.

Type 1 diabetes differs from type 2 diabetes--type 2 is more commonly diagnosed in adulthood, is strongly associated with overweight and obesity and disproportionately affects minority populations. Although patients with type 1 diabetes require externally administered insulin to survive, type 2 diabetes patients may be treated with medications that make their extremities more sensitive to insulin or enhance insulin production or, in some cases, may be treated with insulin itself.

The treatment of patients with type 1 diabetes was revolutionized in 1921 with the discovery of insulin by a group of researchers at the University of Toronto. To this day, insulin therapy continues to save the lives of patients with type 1 diabetes by replacing the essential hormone what their bodies no longer adequately produce. However, insulin therapy, whether through injections or via a pump, is not a cure and it cannot prevent complications. To manage the disease, patients must carefully monitor their food intake and physical activity. They must perform painful finger sticks multiple times a day to draw blood and test their glucose levels. Based on this monitoring, patients often give themselves several shots of insulin a day, or calculate the correct amount of insulin to administer through their insulin pumps. This regimen is not just “once in a while;” it is every day of their lives. As many patients and their parents say; “There is never a day off from diabetes”. Moreover, no matter how vigilant patients are at regulating their blood glucose levels, they can never achieve the fine-tuned regulation provided by a healthy vascular system, which exquisitely senses and responds to insulin needs with precise timing

In 1980 the development of the first animal model of type 1 diabetes that could be used to test drugs for type 1 diabetes; non-obese diabetic (NOD) mouse. Using these NOD mice, doctors from Toronto, the birthplace of insulin discovery, made a revolutionary discovery.

On Dec. 15, 2006, in Canada, a publication in Canada.com reported a Toronto scientist actually appeared to have cured diabetic mice by manipulating the nerves surrounding the insulin-producing islets. Dr. Dosch as early as 1999 concluded that there were surprising similarities between diabetes and multiple sclerosis a central nervous system disease. He suspected a link between the nerves and diabetes. In the article, Dr. Dosch and Dr. Salter used capsaicin, the active ingredient in hot peppers, to kill the pancreatic sensory nerves in mice that had the equivalent of Type 1 diabetes. Once the nerves were deactivated, the islets began producing insulin normally. They had discovered the nerves secrete neuropeptides that are instrumental in the proper functioning of the islets. The University of Calgary and the Jackson Laboratory in Maine found the nerves in diabetic mice were releasing too little of the neuropeptides, resulting in a “vicious cycle” of stress on the islets. In a trial they injected neuropeptide “substance P” in the vascular system of diabetic mice. The islet inflammation cleared up and the diabetes was gone with just one injection. In this study they also discovered that their treatments curbed the insulin resistance that is the hallmark of Type 2 diabetes, and that the insulin resistance is a major factor in Type 1 diabetes, suggesting the two illnesses are quite similar. This research has yet to be tested in clinical trials on humans, but if confirmed it may lead to an eradication of both Type 1 and 2 diabetes.

Solutions to the problem of diabetic disease often involve the use of medications. the most promising appear to be those that can enhance the natural body system called the incretin system, which helps regulate glucose by affecting the beta cells and alpha cells in the vascular system. These prescription medications called dipeptidyl peptidase-4 (DPP-4) inhibitors improve blood sugar control in patients with type 2 diabetes. Through DPP-4 inhibition this new class of drug works when the blood sugar is elevated due to beta-cell dysfunction and uncontrolled production of glucose by the liver due to alpha cell and beta cell dysfunction.

Preferably, these new classes of medications and treatments for diabetes can be more effective when initiated or alternatively combined with the novel use of acoustic shock wave treatments. It is therefore an object of the present invention to treat the extremities of diabetic diagnosed patients or at risk patients of peripheral neuropathy with a regenerating shock wave treatment.

It is also an object of the present invention to provide a shock wave therapy that employs a more effective wave energy transmission, that is both simple to deploy and less target sensitive when compared to reflected focused waves.

It is a further object of the invention to provide a therapeutic treatment of a large target area for subsurface soft extremities of organs such as the vascular system or liver to treat diseases including, but not limited to diabetes.

C. J. Wang discovered that a variety of substances displaying high biological activity are released during and after the application of shock waves to extremity. The production of nitric oxygen (NO), vessel endothelial growth factor (VEGF), bone morphogenetic protein (BMP), and other growth factors have been demonstrated. Furthermore, Maier discovered a decline in the number of small-myelinized neurons after shock wave therapy, an observation that could explain the analgesic effect of shock wave therapy. As a consequence of these findings, the mechanistic model was increasingly relegated to a secondary role and supplanted by a microbiological model explaining the action of shock waves.

In practice the use of ESWT has been a results oriented science wherein a clear and accurate understanding of the actual healing process was neither understood nor fully appreciated. As a result a variety of treatments and uses of ESWT in mammals had heretofore never been tried or attempted or if tried, the outcomes were at best mixed.

A primary factor in the reluctance to use ESWT was that the believed threshold energy requirements were so high that the surrounding extremity would hemorrhage, exhibited by hematomas and bleeding around the treated site. This phenomenon is particularly known in the area of focused emitted waves designed for deep penetration into the patient. US patent publication 2005/0010140 recites the disadvantageous effects of cavitation phenomena can be controlled wherein the shock wave source is connected to a control means which controls the release frequency of shock waves as a function of pulse energy in such a manner that higher pulse energy correlates with lower release frequencies of the shock waves and vice versa. The avoidance of cavitation occurrences would it is postulated result in far less pain for the patient.

In US 2006/0246044 published on Nov. 2, 2006, Andreas Lutz of Dornier Med Tech Systems in Germany disclosed “Methods for Improving Cell Therapy and Extremity Regeneration in Patients With Cardiovascular Disease by Means of Shockwaves”. In this application the use of shock waves is used.

The present invention recognizes the underlying beneficial attributes of ESWT are not now and may never be fully comprehended, however, under a more advanced molecular theory the authors of the present invention postulated a microbiological model suggesting the response mechanism to such treatment.

This model attempts to explain the effect of ESWT by postulating neovascularization of the treated extremity with simultaneous release of diverse growth factors. The enhanced metabolic activity taking place in the presence of these growth factors could be responsible for the healing of the chronically inflamed extremity while the decrease in afferent nerve fibers causes the analgesic effect.

The present inventor sees that ESWT is a highly versatile therapeutic instrument. It can be used as a bioengineering tool to achieve effects such as the production of growth factors or as a surgical instrument to effect an extremely subtle type of denervation. In the field of traumatology, these properties are used primarily to treat fractures with non-union or delayed osseous union. ESWT is also becoming increasingly important for treating the early stages of osteochondritis dissecans. Heretofore the use of ESWT has never been used as a therapeutic instrument in the treatment of diabetes until the attempt to directly treat a pancreas of a diabetic to control insulin production to prevent over production caused by the pancreas as was described in U.S. Pat. No. 7,988,648 granted Aug. 2, 2011. In this prior art patent, the inventors taught treatment methods for stimulating the tissue of a subsurface organ that was part of the incretin system. New data unexpectedly has been discovered suggesting a new preventative treatment for diabetic that is a remote method of controlling blood sugar levels. US patent publication 2017/0296427 published Oct. 19, 2017 entitled “Treatments For Blood Sugar Levels And Muscle Tissue Optimization Using Extracorporeal Acoustic Shock Waves”, disclosed a method of treating red blood cells of a human patient has the steps of activating an acoustic shock wave generator or source to emit acoustic shock waves and subjecting a vascular system containing red blood cells and surrounding muscle tissue peripherally through an extremity of a patient to the acoustic shock waves by stimulating the extremity wherein the extremity is positioned within a path of the emitted shock waves and away from a geometric focal volume or point of the emitted shock waves. This co-pending method which is being incorporated herein in its entirety by reference disclosed a novel way to lower elevated blood glucose levels in diabetic patients.

The present invention refines this technique to combine acoustic shock wave therapy with light therapy to treat even severe cases of peripheral neuropathy as described more fully as follows with first detailed description of shock wave therapeutic methods and then a detailed description of several shock wave devices and apparatus for carrying out the methods.

### SUMMARY OF THE INVENTION

A method of treating a patient with diabetic neuropathy symptoms has the steps of establishing the severity of the neuropathy symptoms, treating the patient with a combination of acoustic shock waves and light therapy. For those patients with symptoms below a threshold level of severity, treating an extremity of those patients with acoustic shock waves followed by near infrared light therapy. For patients at or exceeding the threshold level of severity, treating an extremity of those patients with one or more acoustic shock wave treatments over several weeks, monitoring for a reduction in the severity of neuropathy symptoms to below the threshold level of severity and thereafter following an acoustic shock wave treatment, treating the extremity with a near infrared light therapy.

The step of treating the patient with acoustic shock waves includes the steps of activating an acoustic shock wave generator or source to emit acoustic shock waves through an extremity of the patient; stimulating the sensory nerves of the extremity to rehabilitate and restore function thereby reducing the severity of the diabetic neuropathy symptoms wherein the extremity is positioned in a path of the emitted shock waves. The shock wave generator or source is one of ballistic, radial, piezoelectric, or electrohydraulic. The emitted shock waves are radial, focused, non-focused, planar, nearly planar, convergent or divergent. The acoustic shock waves have a pressure pulse power density in the range of 0.1 to 1.0 mP.

The method further has the step of measuring the patient's blood glucose level as part of the step of establishing the severity of the neuropathy symptoms. The step of establishing the severity of the neuropathy symptoms includes scoring the severity of the neuropathy in an extremity using a modified Toronto Clinical Scoring System (TCSS). In the present inventive method, the patient is tested using the modified (TCS) Toronto Clinical Score wherein temp/cool/vibration/light touch/and heat sensitives are scored. This achieves a quantitative sensitivity test which most physicians might do generally, but in this case scores are established which establish the threshold level of severity which established the treatment protocol. Repeated acoustic treatments and test scores are accomplished for those above the threshold until the score falls at or below the threshold score. The patient is diabetic exhibiting type 1 or type 2 diabetes condition. The extremity is a leg, a foot or can be an arm. The patient has an elevated baseline blood sugar level prior to treating which lowers after treatment, wherein repeating the method periodically a plurality of times over a period of weeks to lower said baseline level of blood sugar. The method can further have the steps of identifying a diabetic at risk patient of neuropathy symptoms, the patient having an at risk baseline blood sugar level; and subjecting the at risk extremity to shock waves to lower said baseline sugar level. The step of identifying an at risk patient includes one or more indications of risk based on family history, genetic disposition, physical condition, or blood or extremity analysis. The step of testing the at risk extremity establishes a measured baseline condition pre shock wave therapy.

Post shockwave therapy testing the blood sugar level allows for comparison to the baseline condition. Preferably, each treated extremity is exposed to a treatment of greater than 500 shock waves and less than 2000 shock waves. Typically, each extremity is exposed to a treatment of about 600 shock waves. Also, each extremity is exposed to a near infrared light over a treatment duration of greater than 10 minutes, preferably about 20 minutes.

Often the symptoms of Chemotherapy-Induced Peripheral Neuropathy (CIPN) are the number one reason why patients have to stop their chemotherapy treatments. In the present invention, a reversal of the neuropathy condition allows the patient to resume potentially life saving chemotherapy. Use of the Toronto Clinical Scoring System (TCSS) is specific and was chosen as the preferred procedure because it reflects the changes of patient's sensitivity each time given. In practice, the present invention can be used to reduce and minimize if not prevent the occurrence of CIPN. Patients having mild symptoms or no symptoms at all can be treated with the inventive method prior to starting chemotherapy and thereafter monitored periodically for symptoms during the entire duration of chemotherapy treatments with repeated acoustic and light therapies as needed. This would avoid any delays or stoppage of chemotherapy. The present invention provides A method of pre-treating a patient prior to or during chemotherapy with diabetic neuropathy symptoms or at risk of having chemotherapy induced peripheral neuropathy comprises the steps of establishing the severity of the neuropathy symptoms; for those patients with symptoms below a threshold level of severity, treating an extremity of those patients with acoustic shock waves followed by near infrared light therapy; for patients at or exceeding the threshold level of severity, treating an extremity of those patients with one or more acoustic shock wave treatments over several weeks, monitoring for a reduction in the severity of neuropathy symptoms to below the threshold level of severity and thereafter following an acoustic shock wave treatment, treating the extremity with a near infrared light therapy; treating the patient with chemotherapy; and monitoring the neuropathy symptoms if any periodically during the duration of chemotherapy.

### Definitions

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude are below 100 ns. The duration of a shock wave is typically below 1-3 micro-seconds (.mu.$) for the positive part of a cycle and typically above some micro-seconds for the negative part of a cycle.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

A “generalized paraboloid” according to the present invention is also a three-dimensional bowl. In two dimensions (in Cartesian coordinates, x and y) the formula yⁿ=2px [with n being not equal to 2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different way, for example: A focused wave will become divergent once it has passed through the focal point. Spherical waves are also included in this definition of divergent waves and have wave fronts with divergent characteristics.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Peripheral Neuropathy” is a disease or degenerative state of the peripheral nerves in which motor, sensory, or vasomotor nerve fibers may be affected and which is marked by muscle weakness and atrophy, pain, and numbness.

### BRIEF DESCRIPTION OF THE DRAWINGS

The invention will be described by way of example and with reference to the accompanying drawings in which:

FIG. 1 illustrates a diabetic patient or a patient at risk of diabetes being treated with a shock wave apparatus in the leg region of an extremity to stimulate the vascular system and surrounding muscle tissue, the patient being oriented face down and lying on his stomach.

FIG. 2 illustrates a diabetic patient or a patient at risk of diabetes being treated with a shock wave apparatus in the arm region of an extremity to stimulate the vascular system and surrounding muscle tissue, the patient being oriented face down and lying on his stomach.

FIG. 3 illustrates a diabetic patient or a patient at risk of diabetes being treated with a shock wave apparatus in the foot region of an extremity to stimulate the vascular system and surrounding muscle tissue, the patient being oriented face down and lying on his stomach.

FIG. 4A is a simplified depiction of a pressure pulse/shock wave (PP/SW) generator with focusing wave characteristics.

FIG. 4B is a simplified depiction of a pressure pulse/shock wave generator with plane wave characteristics.

FIG. 4C is a simplified depiction of a pressure pulse/shock wave generator with divergent wave characteristics.

FIG. 5A is a simplified depiction of a pressure pulse/shock wave generator having an adjustable exit window along the pressure wave path. The exit window is shown in a focusing position.

FIG. 5B is a simplified depiction of a pressure pulse/shock wave generator having an exit window along the pressure wave path. The exit window as shown is positioned at the highest energy divergent position.

FIG. 5C is a simplified depiction of a pressure pulse/shock wave generator having an exit window along the pressure wave path. The exit window is shown at a low energy divergent position.

FIG. 6 is a simplified depiction of an electro-hydraulic pressure pulse/shock wave generator having no reflector or focusing element. Thus, the waves of the generator did not pass through a focusing element prior to exiting it.

FIG. 7A is a simplified depiction of a pressure pulse/shock wave generator having a focusing element in the form of an ellipsoid. The waves generated are focused.

FIG. 7B is a simplified depiction of a pressure pulse/shock wave generator having a parabolic reflector element and generating waves that are disturbed plane.

FIG. 7C is a simplified depiction of a pressure pulse/shock wave generator having a quasi parabolic reflector element (generalized paraboloid) and generating waves that are nearly plane/have nearly plane characteristics.

FIG. 7D is a simplified depiction of a generalized paraboloid with better focusing characteristic than a paraboloid in which n=2. The electrode usage is shown. The generalized paraboloid, which is an interpolation (optimization) between two optimized paraboloids for a new electrode and for a used (burned down) electrode is also shown.

FIG. 8 is a simplified depiction of a pressure pulse/shock wave generator being connected to a control/power supply unit.

FIG. 9 is a simplified depiction of a pressure pulse/shock wave generator comprising a flat EMSE (electromagnetic shock wave emitter) coil system to generate nearly plane waves as well as an acoustic lens. Convergent wave fronts are leaving the housing via an exit window.

FIG. 10 is a simplified depiction of a pressure pulse/shock wave generator having a flat EMSE coil system to generate nearly plane waves. The generator has no reflecting or focusing element. As a result, the pressure pulse/shock waves are leaving the housing via the exit window unfocused having nearly plane wave characteristics.

FIG. 11 is a simplified depiction of a pressure pulse/shock wave generator having a flat piezoceramic plate equipped with a single or numerous individual piezoceramic elements to generate plane waves without a reflecting or focusing element. As a result, the pressure pulse/shock waves are leaving the housing via the exit window unfocused having nearly plane wave characteristics.

FIG. 12 is a simplified depiction of a pressure pulse/shock wave generator having a cylindrical EMSE system and a triangular shaped reflecting element to generate plane waves. As a result, the pressure pulse/shock waves are leaving the housing via the exit window unfocused having nearly plane wave characteristics.

FIG. 13 is a simplified depiction of a pressure pulse/shock wave (PP/SW) generator with focusing wave characteristics shown focused with the focal point or geometrical focal volume being on a substance, the focus being targeted on the location X₀.

FIG. 14 is a simplified depiction of a pressure pulse/shock wave (PP/SW) generator with the focusing wave characteristics shown wherein the focus is located a distance X, from the location X₀ of a substance wherein the converging waves impinge the substance.

FIG. 15 is a simplified depiction of a pressure pulse/shock wave (PP/SW) generator with focusing wave characteristics shown wherein the focus is located a distance X₂ from the mass location X₀ wherein the emitted divergent waves impinge the substance.

FIGS. 16A-16D are color photos of a patient's bottom of a foot 16A and top of a foot 16C before treatments and the same foot after several treatments shown in FIG. 16B bottom and 16D top using the methods of the present invention.

FIG. 17 is a figure of an exemplary light therapy device.

FIG. 18 illustrates an exemplary modified Toronto Clinical Score sheet.

### DETAILED DESCRIPTION OF THE INVENTION

The present invention as described herein provides a unique way to treat peripheral neuropathy symptoms of a diabetic patient's extremities. The treatment involves initial treatments using acoustic shock wave therapy followed by light therapy. What is known about diabetic neuropathy is that it is ischemia issue where oxygen rich blood is not getting to the distal tissue. It's a micro vascular disease, where the thicker blood is not flowing through the smaller capillaries. It's important to note that when diabetics blood glucose levels increase or elevate, the viscosity of the blood gets thicker & doesn't permeate through the smaller blood vessels/capillaries followed by these symptoms, sensory ataxia/balance problems/loss of protections senses are common with this progressive neuro degenerative condition. Neuropathy patients fall much more that the rest of the population. In fact, 1 out of 3 adults over the age of 65 will fall in the next 12 months. It's now become the leading cause of accidental death in people 65 and older.

The inventor has put together strategies to help improve circulation to the tissue and decrease pain. Where there is pain, there is poor blood flow. Both of these devices: ESWT & LLLT have been cleared by the FDA to help improve blood flow & decrease pain. The investor's hypothesis is that when ESWT & LLLT are utilized in combination, the release Nitric Oxide within the lining of the endothelial tissue of the arteries is triggered, causing them to relax or vasodilate. This will in turn improve the blood flow to that targeted treatment area. Successful clinical evidence appears to confirm this therapy.

An important aspect of this invention is the physician preferably establishes a threshold severity level of the neuropathy symptoms. At extremely low severity levels, light therapy alone is a sufficient treatment. However, at moderate to high severity levels, as defined by the protocols used for measuring severity, the use of light therapy is relatively ineffective. Heretofore, this left the patient with little hope for overcoming the condition and running the risk of amputation int eh severe outcomes common for diabetics.

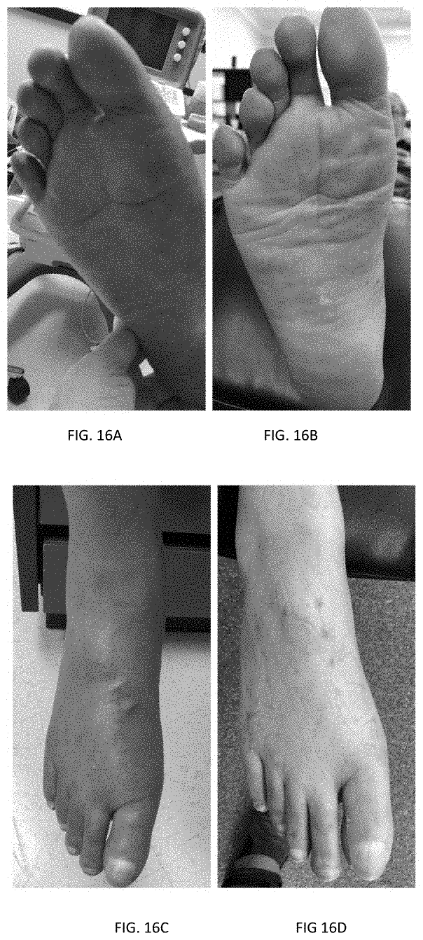

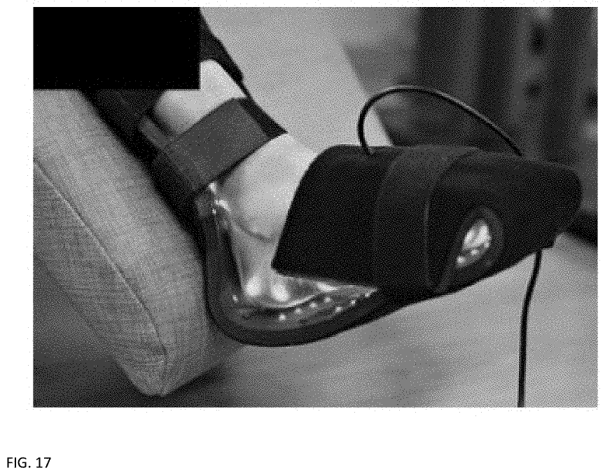

To best understand the procedure, reference is made to the photographs of FIGS. 16A and 16B of the bottom of a foot and FIGS. 16C and 16D of the top of the foot prior to treatment. As shown in figure FIGS. 16A and 16C, the purplish color of the dermis shows the peripheral damage. After several treatments using the protocol of the present invention, the skin (dermis) color is returned to normal and the symptoms of peripheral neuropathy damage are gone as shown in FIGS. 16B and 16D. An interesting and valuable outcome of the present invention is once the symptoms are resolved and re-established, it is established that a risk of a recurrence could occur and therefore preventive treatments can be given annually or semi-annually.

This is more than precautionary as the inventors have proven cases that exceed the maximum severity threshold will require several repeated treatments of acoustic shock wave therapy to lower the severity threshold into a range where the combination of acoustic and light therapy will be effective.

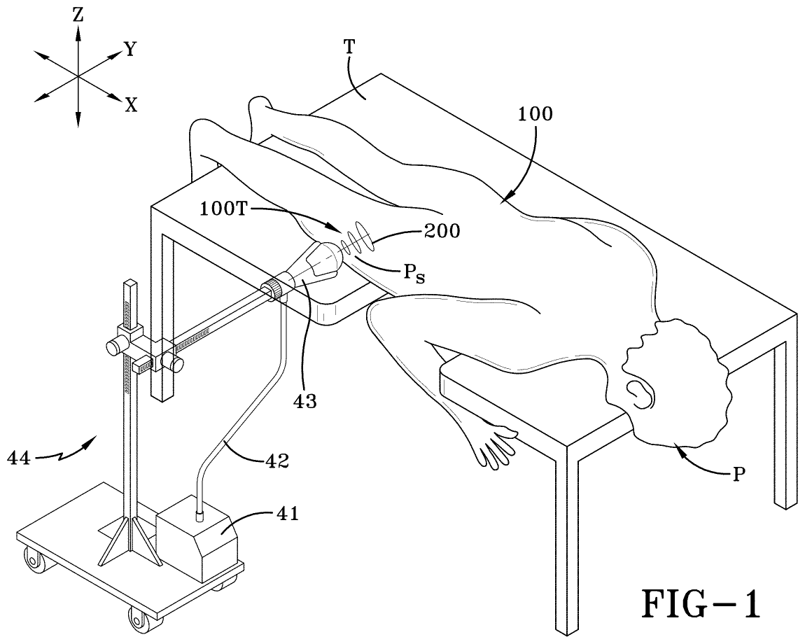

With reference to FIG. 1, the patient P who has either been diagnosed with a diabetic neuropathy symptom or is at risk of contracting peripheral neuropathic symptoms related to diabetes is positioned on a table T preferably face down lying on the stomach. A shock wave applicator head 43 is brought into contact with the skin Ps of a leg 100T preferably an acoustic gel is used to enhance the transmission of the shock waves 200 through the body down to the subsurface extremity 100 containing a portion of the vascular system 101 surrounded by muscle tissue 102. The shock wave applicator head 43 is connected via cabling 42 to a power generating unit 41 as shown. The shock wave applicator head 43 can be attached rigidly to a fixture or stand 44 as illustrated or alternatively can be hand held and manipulated across the skin Ps to drive the shock waves 200 in the direction the shock wave head 43 is pointed.

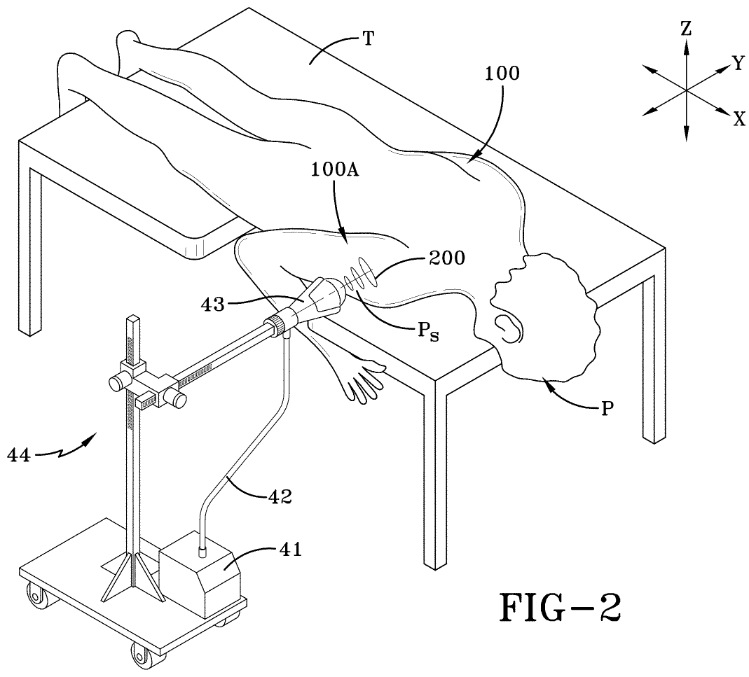

With reference to FIG. 2, the patient P's body is shown with the applicator directly above an arm 100A containing a portion of the vascular system 101 surrounded by muscle tissue 102. As illustrated the vascular system 101 is being bombarded with shock waves 200 that are emitted from the lens 17 directly into the patient P to provide the therapeutic treatment of acoustic shock waves 200 to the extremity 100.

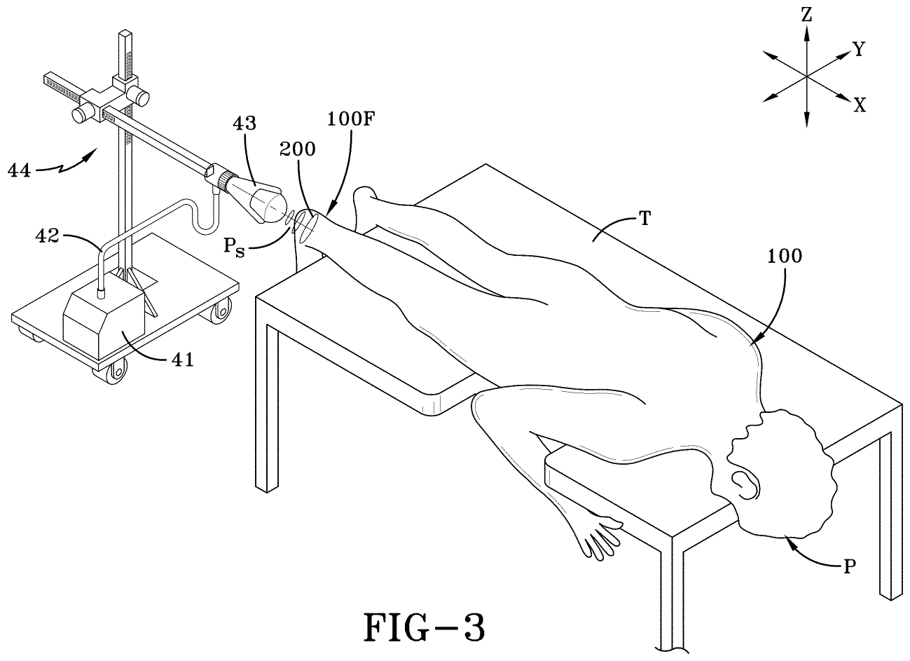

With reference to FIG. 3, the patient P's body is shown with the applicator directly above a foot 100F containing a portion of the vascular system 101 surrounded by muscle tissue 102. As illustrated the vascular system 101 is being bombarded with shock waves 200 that are emitted from the lens 17 directly into the patient P to provide the therapeutic treatment of acoustic shock waves 200 to the extremity 100. Numerous large blood vessels 122 lie within the extremity 100. When these nerve cells are irritated or not properly producing neuropeptides, it is possible to shut down the islets such that they will no longer properly produce insulin. This is a condition commonly referred to as diabetes and can occur in type 1 or type 2 diabetes. When this condition occurs, it has been determined that shutting the nerve cells down for a period of time will enable the islets to continue to produce insulin in a normal fashion. It has therefore been determined that the analgesic effect of shock waves 200 when bombarding the vascular system 101 can be used to at least partially deactivate the nerve cells surrounding the islet such that these islets can begin to produce insulin properly. After a treatment with shock waves 200 it has further been determined that the damaged nerve cells in the incretin nerve system surrounding the islets 120 can be healed and stimulated to properly secrete neuropeptides which when properly secreted further enhance the ability of the islets to produce insulin normally. After treatment with shock waves 200 the chronic inflammation commonly associated with the vascular system and these extremities 100 can be reduced dramatically indicating that the extremity 100 is being healed in such a fashion that the nerves surrounding are no longer irritated or sensitized and that the entire pancreatic system can now perform properly. The common pain associated with the pancreatic condition can be alleviated in this way and the proper functioning of the vascular system 101 can be stimulated such that the diabetic condition can be eliminated or greatly reduced and the blood cells of the vascular system 101 can operate normally with normal levels of blood sugar. This condition can be conducted on a person already experiencing diabetic indications of type 1 or type 2 or can be used on people with known risk of diabetic conditions. It is preferred that the treatment be used with non focused shock waves to eliminate or minimize hemorrhaging or focused shock waves can be used wherein the wave pattern does not have the focal point generated on the pancreatic extremity 100, otherwise this can create hemorrhaging and potential damage to the vascular system 101 if the intensity level of the focused shock wave is too intense. For these reasons it is preferable to use a radial source or the divergent or non-planar or planar shock waves for the treatment of a diabetic vascular system 101. These lower energy, lower amplitude shock waves described hereinafter can provide the beneficial effects without creating any trauma to the vascular or muscular skeletal systems, furthermore these treatments can be done on an outpatient basis as will be described.

A case series by Kenneth Craig Vincent, Medical Director-Kompass Health Associates, Auckland, New Zealand, attempting to improve skeletal muscle function in the older adult population derived some interesting complimentary data. The application of shockwave treatment (SWT) utilizing a DermaGold-100 (TRT LLC, USA) not only improved muscle mass, balance, and stability across the 10 trial subjects, but simultaneously indicated that blood sugar levels of three (3) of the diabetic subjects noticed an improvement in both fasting (Table 1) and postprandial (Table 2) blood sugar levels after 3 sessions of SWT.

The treatment protocol involved the application of SWT onto the hamstrings, gastrocnemius/soleus complex, and the planter aspect of the foot. Three session of SWT were applied onto to each subject over a one week interval.

TABLE 1
Fasting Blood Sugar levels taken by patient utilizing personal glucometer.
Readings are based on the daily average over each week.

| | | Wk 2 | Wk 4 | Wk 8 |
|---|---|---|---|---|
| Fasting | | Post | Post | Post |
| Blood Sugar | Baseline | SWT | SWT | SWT |
| Subject 1 (62 yr old) | 128 mg/dl | 126 mg/dl | 122 mg/dl | 116 mg/dl |
| Subject 2 (56 yr old) | 122 mg/dl | 119 mg/dl | 117 mg/dl | 112 mg/dl |
| Subject 3 (58 yr old) | 126 mg/dl | 123 mg/dl | 121 mg/dl | 118 mg/dl |

TABLE 2
Blood sugar level 2 hours postprandial levels taken
by patient utilizing personal glucometer. Readings are
based on the daily average over each week.

| | | Wk 2 | Wk 4 | Wk 8 |
|---|---|---|---|---|
| Postprandial | | Post | Post | Post |
| Blood Sugar | Baseline | SWT | SWT | SWT |
| Subject 1 (62 yr old) | 163 mg/dl | 159 mg/dl | 157 mg/dl | 153 mg/dl |
| Subject 2 (56 yr old) | 158 mg/dl | 158 mg/dl | 155 mg/dl | 150 mg/dl |
| Subject 3 (58 yr old) | 161 mg/dl | 157 mg/dl | 154 mg/dl | 151 mg/dl |

These figures suggest that the increase in skeletal muscle mass and activity would increase metabolic demand and simultaneously increase blood sugar uptake, improving both fasting and postprandial blood sugar levels in diabetic patients. It is therefore plausible to hypothesize that application of an increased number of SWT impulses over the skeletal muscle of the lower extremity could help improve the control of blood sugar levels in diabetics.

Accordingly, a periodic treatment regimen of emitting 500 or more shock wave pressure pulses, preferably about 1500 pressure pulses at a low pulse energy of 0.1 mJ/mm² or higher up to 1.0 mJ/mm², preferably about 0.3 to 0.5 mJ/mm² over a period of weeks on the extremity 100 will remotely improve the patient's high baseline blood sugar levels to approach, if not achieve, normal blood sugar levels.

Herein lies an incredible opportunity to treat patients with a combination of acoustic shock waves to lower the threshold level of the symptoms to an amount wherein light therapy can be successfully used. In the past, the treatment using light therapy devices were ineffective at high severity levels. Now, the threshold levels can be lowered as well as the elevated blood glucose levels after acoustic therapy to make it possible to successfully use light therapy like near infrared devices when combined with acoustic therapy.

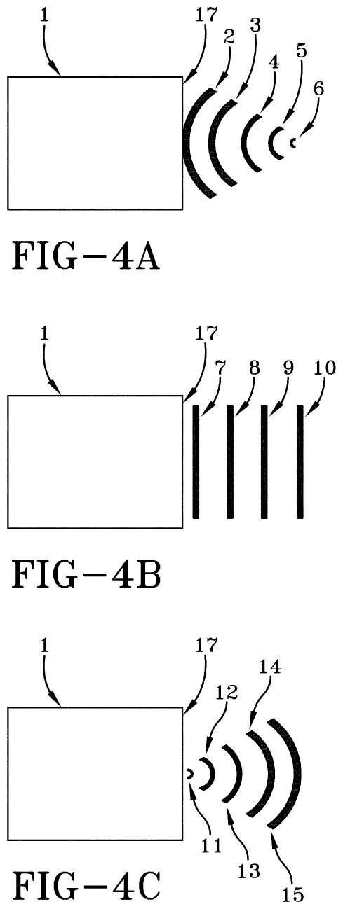

The following description of the proper amplitude and pressure pulse intensities of the shock waves 200 are provided below along with a description of how the shock waves actually function and have been taken from the co-pending application of the present inventors and replicated herein as described below. For the purpose of describing the shock waves 200 were used as exemplary and are intended to include all of the wave patterns discussed in FIGS. 4A-15 as possible treatment patterns.

This method of treatment has the steps of, locating a treatment site, generating either convergent diffused or far-sighted focused shock waves or unfocused shock waves, of directing these shock waves to the treatment site; and applying a sufficient number of these shock waves to induce activation of one or more growth factor thereby inducing or accelerating healing.

The unfocused shock waves can be of a divergent wave pattern or near planar pattern preferably of a low peak pressure amplitude and density. Typically, the energy density values range as low as 0.000001 mJ/mm² and having a high end energy density of below 1.0 mJ/mm², preferably 0.20 mJ/mm² or less. The peak pressure amplitude of the positive part of the cycle should be above 1.0 and its duration is below 1-3 microseconds.

The treatment depth can vary from the surface to the full depth of the human or animal torso and the treatment site can be defined by a much larger treatment area than the 0.10-3.0 cm² commonly produced by focused waves. The above methodology is particularly well suited for surface as well as sub-surface soft extremity treatments.

While one of the benefits of the non-invasive nature of this treatment relates to reducing patient recovery and healing time, the fact that the treatments can be delivered at dosages well below the threshold of pain means that no local or general anesthesia is typically required as a consequence of the treatment. This alone significantly reduces any risk factors or complications associated with pain management during the procedure. The treatments further can reduce the need for a regiment of chemical or drug therapies before or after exposure to this shock wave therapy. Alternatively, ESWT can be used in conjunction with chemical or drug therapies to increase the cellular response permitting an opportunity to lower dosages of such chemicals or drugs while increasing the therapeutic efficiency. This is a particularly useful tool for the physician whose patient is elderly, a smoker or with an immune system deficiency which would complicate if not make unavailable more traditional invasive surgical procedures. In fact, the above methodology proposed in this patent may be the first if not only choice of treatment available for patients in this class wherein heretofore conventional procedures were deemed too risky.

A further clinical benefit of the above methodology is that the procedure can be done either as an outpatient treatment or at a doctor's office assuming the patient's condition does not otherwise require hospitalization.

The stimulation of growth factors and activation of healing acceleration is particularly valuable to elderly patients and other high risk factor subjects.

Even more striking as mentioned earlier, early prevention therapies can be employed to stimulate extremity or organ modeling to be maintained within acceptable ranges prior to a degeneration occurring. This is extremely valuable in the prevention of diabetes or heart disease for example. The methods would be to identify at risk patients based on family history or genetic disposition, physical condition, etc. and subjecting that patient to therapeutic shock wave therapy for the purpose of stimulating extremity repair effectively remodeling the patient's susceptible organ to be within accepted functional parameters. The objective being to preventively stimulate cellular extremity repairs to pre-emptively avoid a degenerative condition from occurring which may require invasive surgical procedures.

FIG. 4A is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The diseased organ is generally located in or near the focal point which is located in or near position 6. At position 17 a water cushion or any other kind of exit window for the acoustical energy is located.

FIG. 4B is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a point source (e.g. electrode) that is located in the focal point of the paraboloid. The waves will be transmitted into the patient's body via a coupling media such as, e.g., ultrasound gel or oil and their amplitudes will be attenuated with increasing distance from the exit window 17.

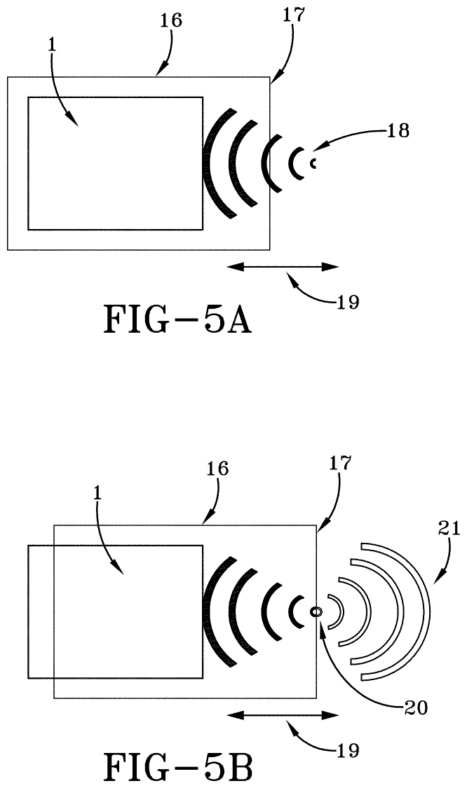

FIG. 4C is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In FIG. 4C the pressure pulse source may be a point source, that is, the pressure pulse may be generated by an electrical discharge of an electrode under water between electrode tips. However, the pressure pulse may also be generated, for example, by an explosion. The divergent characteristics of the wave front may be a consequence of the mechanical setup shown in FIG. 5B.

FIG. 5A is a simplified depiction of a pressure pulse/shock wave generator (shock wave head) according to the present invention having an adjustable or exchangeable (collectively referred to herein as “movable”) housing around the pressure wave path. The apparatus is shown in a focusing position. FIG. 5A is similar to FIG. 4A but depicts an outer housing (16) in which the acoustical pathway (pressure wave path) is located. In a preferred embodiment, this pathway is defined by especially treated water (for example, temperature controlled, conductivity and gas content adjusted water) and is within a water cushion or within a housing having a permeable membrane, which is acoustically favorable for the transmission of the acoustical pulses. In certain embodiments, a complete outer housing (16) around the pressure pulse/shock wave generator (1) may be adjusted by moving this housing (16) in relation to, e.g., the focusing element in the generator. However, as the person skilled in the art will appreciate, this is only one of many embodiments of the present invention. While the figure shows that the exit window (17) may be adjusted by a movement of the complete housing (16) relative to the focusing element, it is clear that a similar, if not the same, effect can be achieved by only moving the exit window, or, in the case of a water cushion, by filling more water in the volume between the focusing element and the cushion. FIG. 5A shows the situation in which the arrangement transmits focused pressure pulses.

FIG. 5B is a simplified depiction of the pressure pulse/shock wave generator (shock wave head) having an adjustable or exchangeable housing around the pressure wave path with the exit window 17 being in the highest energy divergent position. The configuration shown in FIG. 5B can, for example, be generated by moving the housing (16) including the exit window (17), or only the exit window (17) of a water cushion, towards the right (as shown in the Figure) to the second focus f2 (20) of the acoustic waves. In a preferred embodiment, the energy at the exit window will be maximal. Behind the focal point, the waves may be moving with divergent characteristics (21).

FIG. 5C is a simplified depiction of the pressure pulse/shock wave generator (shock wave head) having an adjustable or exchangeable housing around the pressure wave path in a low energy divergent position. The adjustable housing or water cushion is moved or expanded much beyond f2 position (20) so that highly divergent wave fronts with low energy density values are leaving the exit window (17) and may be coupled to a patient's body. Thus, an appropriate adjustment can change the energy density of a wave front without changing its characteristic.

This apparatus may, in certain embodiments, be adjusted/modified/or the complete shock wave head or part of it may be exchanged so that the desired and/or optimal acoustic profile such as one having wave fronts with focused, nearly plane or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In certain embodiments, the change of the distance of the exit acoustic window can be accomplished by a sliding movement. However, in other embodiments of the present invention, in particular, if mechanical complex arrangements, the movement can be an exchange of mechanical elements.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the present invention provides versatility, the person skilled in the art will appreciate that apparatuses that only produce waves having, for example, nearly plane characteristics, are less mechanically demanding and fulfill the requirements of many users.

As the person skilled in the art will also appreciate that embodiments shown in drawings 4A-4C and 5A-5C are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

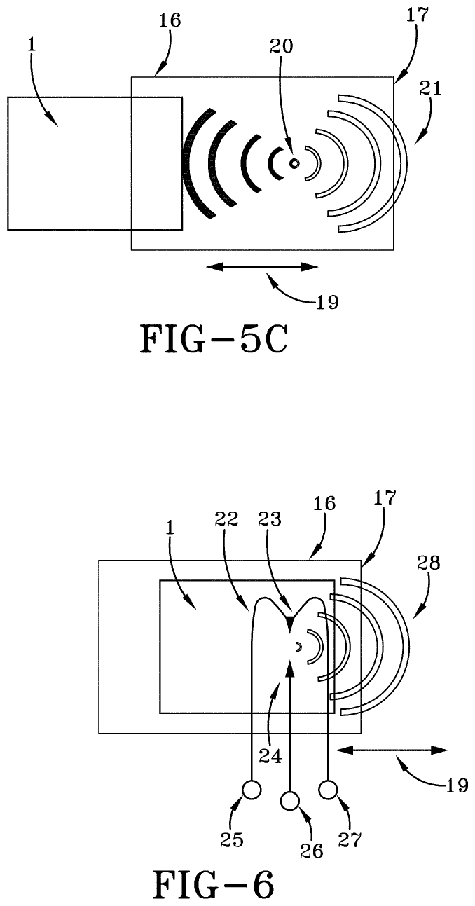

FIG. 6 is a simplified depiction of the pressure pulse/shock wave apparatus having no focusing reflector or other focusing element. The generated waves emanate from the apparatus without coming into contact with any focusing elements. FIG. 6 shows, as an example, an electrode as a pressure pulse generating element producing divergent waves (28) behind the ignition point defined by a spark between the tips of the electrode (23, 24).

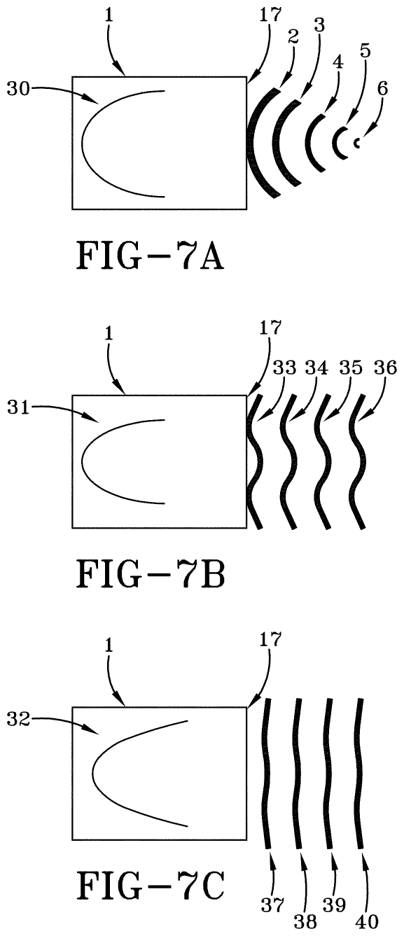

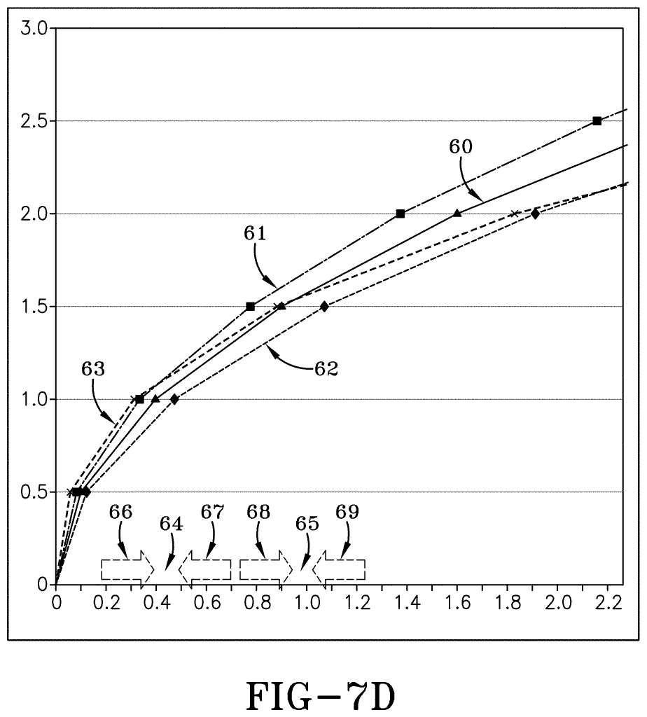

FIG. 7A is a simplified depiction of the pressure pulse/shock wave generator (shock wave head) having as focusing element an ellipsoid (30). Thus, the generated waves are focused at (6).

FIG. 7B is a simplified depiction of the pressure pulse/shock wave generator (shock wave head) having as a focusing element a paraboloid (y²=2px). Thus, the characteristics of the wave fronts generated behind the exit window (33, 34, 35, and 36) are disturbed plane (“parallel”), the disturbance resulting from phenomena ranging from electrode burn down, spark ignition spatial variation to diffraction effects. However, other phenomena might contribute to the disturbance.

FIG. 7C is a simplified depiction of the pressure pulse/shock wave generator (shock wave head) having as a focusing element a generalized paraboloid (y¹¹=2px, with 1.2<n<2.8 and n≈2). Thus, the characteristics of the wave fronts generated behind the exit window (37, 38, 39, and 40) are, compared to the wave fronts generated by a paraboloid (y²=2px), less disturbed, that is, nearly plane (or nearly parallel or nearly even (37, 38, 39, 40)). Thus, conformational adjustments of a regular paraboloid (y²=2px) to produce a generalized paraboloid can compensate for disturbances from, e.g., electrode burn down. Thus, in a generalized paraboloid, the characteristics of the wave front may be nearly plane due to its ability to compensate for phenomena including, but not limited to, burn down of the tips of the electrode and/or for disturbances caused by diffraction at the aperture of the paraboloid. For example, in a regular paraboloid (y²=2px) with p=1.25, introduction of a new electrode may result in p being about 1.05. If an electrode is used that adjusts itself to maintain the distance between the electrode tips (“adjustable electrode”) and assuming that the electrodes burn down is 4 mm (z=4 mm), p will increase to about 1.45. To compensate for this burn down, and here the change of p, and to generate nearly plane wave fronts over the life span of an electrode, a generalized paraboloid having, for example n=1.66 or n=2.5 may be used. An adjustable electrode is, for example, disclosed in U.S. Pat. No. 6,217,531.

FIG. 7D shows sectional views of a number of paraboloids. Numeral 62 indicates a paraboloid of the shape y²=2px with p=0.9 as indicated by numeral 64 at the x axis which specifies the p/2 value (focal point of the paraboloid). Two electrode tips of a new electrode 66 (inner tip) and 67 (outer tip) are also shown in the Figure. If the electrodes are fired and the tips are burning down the position of the tips change, for example, to position 68 and 69 when using an electrode which adjusts its position to compensate for the tip burn down. In order to generate pressure pulse/shock waves having nearly plane characteristics, the paraboloid has to be corrected in its p value. The p value for the burned down electrode is indicated by 65 as p/2=1. This value, which constitutes a slight exaggeration, was chosen to allow for an easier interpretation of the Figure. The corresponding paraboloid has the shape indicated by 61, which is wider than paraboloid 62 because the value of p is increased. An average paraboloid is indicated by numeral 60 in which p=1.25 cm. A generalized paraboloid is indicated by dashed line 63 and constitutes a paraboloid having a shape between paraboloids 61 and 62. This particular generalized paraboloid was generated by choosing a value of n 2 and a p value of about 1.55 cm. The generalized paraboloid compensates for different p values that result from the electrode burn down and/or adjustment of the electrode tips.

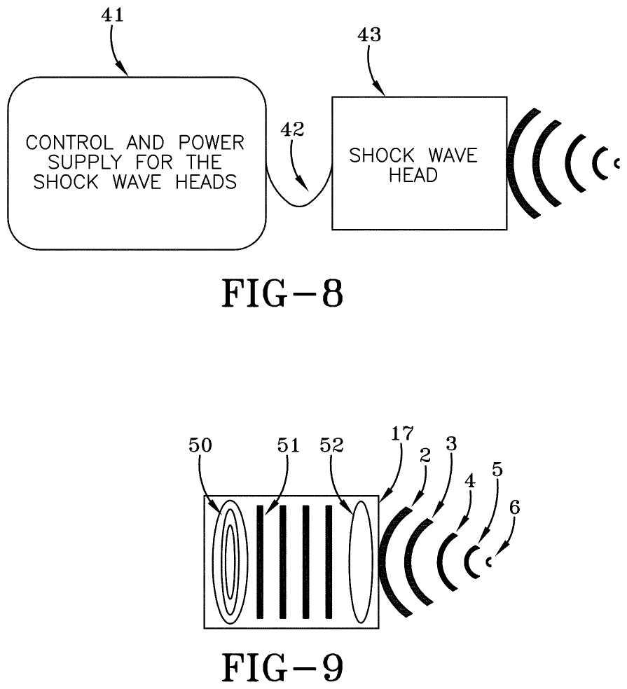

FIG. 8 is a simplified depiction of a set-up of the pressure pulse/shock wave generator (43) (shock wave head) and a control and power supply unit (41) for the shock wave head (43) connected via electrical cables (42) which may also include water hoses that can be used in the context of the present invention. However, as the person skilled in the art will appreciate, other set-ups are possible and within the scope of the present invention.

FIG. 9 is a simplified depiction of the pressure pulse/shock wave generator (shock wave head) having an electromagnetic flat coil 50 as the generating element. Because of the plane surface of the accelerated metal membrane of this pressure pulse/shock wave generating element, it emits nearly plane waves which are indicated by lines 51. In shock wave heads, an acoustic lens 52 is generally used to focus these waves. The shape of the lens might vary according to the sound velocity of the material it is made of. At the exit window 17 the focused waves emanate from the housing and converge towards focal point 6.

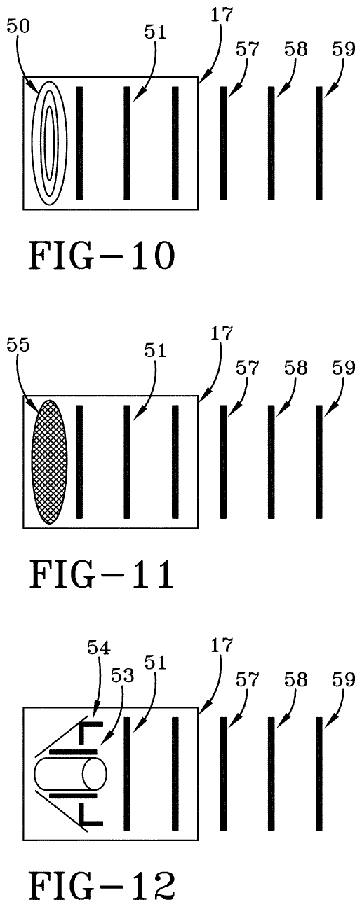

FIG. 10 is a simplified depiction of the pressure pulse/shock wave generator (shock wave head) having an electromagnetic flat coil 50 as the generating element. Because of the plane surface of the accelerated metal membrane of this generating element, it emits nearly plane waves which are indicated by lines 51. No focusing lens or reflecting lens is used to modify the characteristics of the wave fronts of these waves, thus nearly plane waves having nearly plane characteristics are leaving the housing at exit window 17.

FIG. 11 is a simplified depiction of the pressure pulse/shock wave generator (shock wave head) having an piezoceramic flat surface with piezo crystals 55 as the generating element. Because of the plane surface of this generating element, it emits nearly plane waves which are indicated by lines 51. No focusing lens or reflecting lens is used to modify the characteristics of the wave fronts of these waves, thus nearly plane waves are leaving the housing at exit window 17. Emitting surfaces having other shapes might be used, in particular curved emitting surfaces such as those shown in FIGS. 7A to 7C as well as spherical surfaces. To generate waves having nearly plane or divergent characteristics, additional reflecting elements or lenses might be used. The crystals might, alternatively, be stimulated via an electronic control circuit at different times, so that waves having plane or divergent wave characteristics can be formed even without additional reflecting elements or lenses.

FIG. 12 is a simplified depiction of the pressure pulse/shock wave generator (shock wave head) comprising a cylindrical electromagnet as a generating element 53 and a first reflector having a triangular shape to generate nearly plane waves 54 and 51. Other shapes of the reflector or additional lenses might be used to generate divergent waves as well.

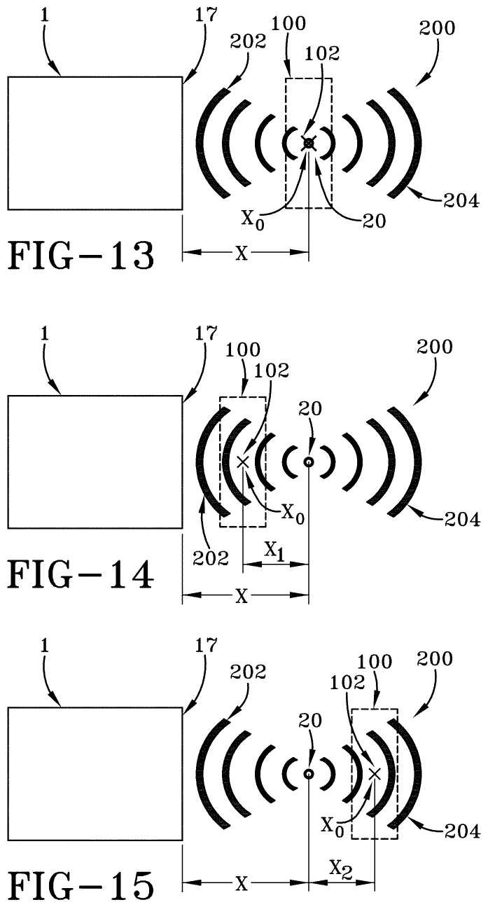

With reference to FIGS. 13, 14 and 15 a schematic view of a shock wave generator or source 1 is shown emitting a shock wave front 200 from an exit window 17. The shock wave front 200 has converging waves 202 extending to a focal point or focal geometric volume 20 at a location spaced a distance X from the generator or source 1. Thereafter the wave front 200 passes from the focal point or geometric volume 20 in a diverging wave pattern as has been discussed in the various other FIGS. 4-12 generally.

With particular reference to FIG. 13 extremity of a subsurface organ 100 is shown generally centered on the focal point or volume 20 at a location X₀ within the extremity of a subsurface organ 100. In this orientation the emitted waves are focused and thus are emitting a high intensity acoustic energy at the location X₀. This location X₀ can be anywhere within or on the extremity of a subsurface organ. Assuming the extremity of a subsurface organ 100 is an extremity having a mass 102 at location X₀ then the focus is located directly on the mass 102. In one method of treating a tumor or any other type mass 102 these focused waves can be directed to destroy or otherwise reduce the mass 102.

With reference to FIG. 14, the substance 100 is shifted a distance X toward the generator or source 1. The extremity of a subsurface organ 100 at location X₀ being positioned a distance X-X₁ from the source 1. This insures the extremity of a subsurface organ 100 is impinged by converging waves 202 but removed from the focal point 20. When the extremity of a subsurface organ 100 is extremity this bombardment of converging waves 202 stimulates the cells activating the desired healing response as previously discussed, this also is one of the preferred methods to treat an inflamed diabetic pancreatic extremity.

With reference to FIG. 15, the extremity of a subsurface organ 100 is shown shifted or located in the diverging wave portion 204 of the wave front 200. As shown X₀ is now at a distance X₂ from the focal point or geometric volume 20 located at a distance X from the source 1. Accordingly X₀ is located a distance X+X₂ from the source 1. As in FIG. 10 this region of diverging waves 204 can be used to stimulate the substance 100 which when the substance is a cellular extremity stimulates the cells to produce the desired healing effect or response, this is also one of the preferred methods.

As shown the use of these acoustic wave forms can be used separately or in combination to achieve the desired therapeutic effect.

Furthermore, such acoustic wave forms can be used in combination with drugs, chemical treatments, irradiation therapy or even physical therapy and when so combined the stimulated cells will more rapidly assist the body's natural healing response.

In the present invention, the acoustic shock waves are used in combination with light therapy treatments to remedy peripheral neuropathic conditions.

The present invention provides an apparatus for an effective treatment of indications, which benefit from low energy pressure pulse/shock waves having nearly plane or even divergent characteristics. For the treatment of those indications, the procedure to locate the area to which the pressure pulses/shock waves are applied often needs to be less accurate than, e.g., when kidney stones are destroyed with focused waves. In fact, sometimes the knowledge of the physique of the subject to be treated is sufficient, so that imaging devices like ultrasound, x-ray or similar, as they are known from devices used in the destruction of kidney stones, may not be required. The area of the focal point/focus volume can be enlarged by reducing the focusing or even by eliminating it all together by using an apparatus according to the present invention which produces waves having wave fronts with nearly plane or divergent characteristics.

With an unfocused wave having nearly plane wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000001 mJ/mm² In a preferred embodiment, those low end values range between 0.1-0.001 mJ/mm² With these low energy densities, side effects are reduced and the dose application is much more uniform. Additionally, the possibility of harming surface extremity is reduced when using an apparatus of the present invention that generates waves having nearly plane or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output.

The treatment of the above mentioned diabetic indications are believed to be a first time use of acoustic shock wave therapy in combination with light therapy. None of the work done to date has treated the above mentioned indications with convergent, divergent, planar or near-planar acoustic shock waves of low energy.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

Dr.'s Matthew M. Diduro and Noah D. Marchese co-authored a study entitled, “Clinical Experience using NIR light therapy in subjects with Type II diabetes complaining of neuropathy”. This subject matter being incorporated by reference in its entirety.

The purpose of this case report was to evaluate the effect of near infrared light therapy treatments on pain and other symptoms of diabetic peripheral neuropathy (DPN).

The severity of neuropathy was assessed in 152 subjects in an outpatient pain clinic. Pain (VAS), a peripheral neuropathy questionnaire (PNQ), a restless leg syndrome questionnaire (RLSQ) and the Toronto Clinical Scoring System (TCSS) were used to determine the severity of symptoms. Following a mean of 18±7 light therapy treatments (20 minutes, 3 times/week) on the lower extremities, the tests were repeated. Data were analyzed with a statistical two-sided one-sample t-test that the change from baseline in response to light therapy was zero. Data are presented as mean±standard deviation.

The average age in the subjects was 64 ±12 years. Baseline pain was 7.8±1.5, PNQ was 58.7±21.2%, RLSQ was 58.9±21.1%, TCSS Left Leg was 37.0±

±21.4% and TCSS Right Leg was almost identical at 37.1±21.8%. These measures indicated that the subjects had diabetic neuropathy. Following treatments with light therapy, pain decreased by 4.4±2.2 (P<0.0001), PNQ improved by 13.5±20.9% (P<0.0001), RLSQ improved by 13.1±22.2% (P<0.0001). Similarly, the TCSS increased by 28.7±19.9% on the right leg and by 29.7±19.6% on the left leg (both P<0.0001).

The symptoms of neuropathy improved so rapidly in some subjects that they were able to cease treatments in less than 5 weeks. Other subjects whose progress was much slower required 8 weeks or, in a few cases, more than 8 weeks to realize significant reduction of most of their neuropathy symptoms. This is likely due to the complexity of Type II diabetes and associated comorbidities. Some light therapy devices may improve the symptoms of DPN and improve quality of life.

Some of the symptoms of diabetic neuropathy are numbness, a reduced ability to feel pain or temperature changes, a tingling or burning sensation, sharp pains or cramps, increased sensitivity to touch—for some people even the weight of a bed sheet can be agonizing—muscle weakness, loss of reflexes especially in the ankle, loss of balance and coordination, serious foot problems, such as ulcers, infections, deformities, and bone and joint pain (1).

Despite tight control of blood sugar, exercise and improvements in life style, these symptoms can develop, persist or worsen. There are no treatments for diabetic neuropathy although some pharmaceutical products are FDA approved and may be helpful for mitigating the symptom of diabetic nerve pain.

Persistently elevated blood glucose (EBG) underlies the pathophysiology of diabetic neuropathy and its symptoms (2). People with type II diabetes suffer from various degrees of retinopathy, coronary heart and kidney disease, gastroparesis, and stroke. Each of these can be considered to be the result of vascular injury caused, at least in part, by EBG. Simply put, low, subnormal blood flow deprives cells, including nerves, of the oxygen and glucose necessary for ATP production in mitochondria. ATP is required by cells to regulate membrane potential. Depolarized nerves send pain signals to the brain or fail to properly detect sensations.

Clearly, if one could temporarily or chronically improve blood flow, then ATP production would recover toward normal as glucose and oxygen increased at the cellular level. Pain signals related to depolarized nerves should diminish as membrane potential returns toward normal.

In fact, improved circulation to nerves might minimize or eliminate many, if not most, symptoms of diabetic neuropathy. There are several light therapy devices, (LED or laser based), cleared by the FDA for increasing circulation that should mitigate the painful symptoms in subjects with diabetic neuropathy.

This report summarizes clinical outcomes in diabetic subjects treated at one of our clinics. The subjects had previously received one or more medications but were dissatisfied with the outcomes related to their pain and/or other neuropathy symptoms or the side effects.

Subjects who presented to our clinic with the symptoms of neuropathy were given a complete physical. A thorough medical history was obtained; if the person was diabetic (HgAlc >5.5) a Pain Self-Assessment Form was completed (Visual Analogue Scale: 0-10) as well as a Peripheral Neuropathy Questionnaire (PNQ) in which answers were converted to percentages. 0-20%=zero to mild peripheral neuropathy, 21-40%=moderate peripheral neuropathy, 41-60%=severe peripheral neuropathy, above 61%=crippling level of peripheral neuropathy; i.e. wheel chairs, bed bound, crippled. Restless Leg Syndrome (RLS) is prevalent in type II Diabetes (3) so a Restless Leg Syndrome (RLS) Questionnaire was also completed. These were also converted to percentages.

We also scored the severity of neuropathy in each leg using the Toronto Clinical Scoring System (TCSS). The maximum score in a patient with no symptoms is 74 (100%). The following are the results of the severity of neuropathy in 152 subjects. The subjects were then offered treatment with an FDA cleared near infrared LED device (HealthLight PN LLC, Reno, Nev. 89523). The protocol was explained, and informed consent was obtained. Treatments were on the foot and calf of each leg for 20 minutes 3 times/week. The PN and RLS symptoms and the Toronto scores were re-assessed after 6 treatments to determine if there were any improvements in the interim period. These were re-evaluated again following the completion of all treatments.

Data were analyzed with a statistical two-sided one-sample t-test that the change from baseline in response to light therapy was zero. Data are presented as mean±standard deviation.

Type II diabetes is more prominent in adults. Average age of our subjects was 64±12 years. Baseline pain was high; 7.8±1.5, PNQ was low; 58.7±21.2%, RLSQ was also low; 58.9±21.1%. The TCSS Left Leg was Low; 37.0±21.4% and TCSS Right Leg was almost identical at 37.1±21.8%. These subjects exhibited the symptoms of neuropathy.

Following a mean of 18 treatments, pain decreased by 4.4±2.2 (P<0.0001), PNQ improved by 13.5±20.9% (P<0.0001) and the RLSQ improved by 13.2±22.2% (P<0.0001). Similarly, the TCSS increased by 28.7±19.9% on the right leg and by 29.7±19.6% on the left leg (both P<0.0001). The symptoms of neuropathy improve so rapidly in some subjects that they were able to cease treatments in less than 5 weeks. Other subjects whose progress was much slower required 8 weeks or, in a few cases, more than 8 weeks to have resolution most of their neuropathy symptoms. This is likely due to the complexity of Type II diabetes and comorbidities.

The box plot for pain is presented in the diagrams. The line in the box is the median. The closer to the middle of the box the median is, the less asymmetric the data are. If the median is in the upper part of the box, the data are skewed to the top and have a tail to the bottom of the graph. If the median is in the lower part of the box, then the data are skewed to the bottom of the figure with a long tail to the top. Asterisks that appear below or above the lines extending from the box can be considered outliers.

An analysis was done by linear regression to see if there was a trend in change from baseline with increasing numbers of treatments. The mean number of treatments was 18.05 (SD=7.334) with median of 15, (-5 weeks); range 7, 44. The change from baseline Pain or PNQ did not have a relationship with the number of treatments with P=0.668 and P=0.294, respectively. This means that in most subjects treated 3 times a week for 20 minutes each time, the lowest pain level achievable would be approximately 3.7 and that more treatments may not improve this or the PNQ symptoms.

However, RLSQ, TCSS Left, and TCSS Right did have a statistically significant relationship with the number of treatments with P=0.006, P=0.021, and P=0.007, respectively. The change in RLSQ was improved by about 0.8% for each additional treatment. The TCSS Left was increased by about 0.57% for each additional treatment and TCSS Right was increased by about 0.65% for each additional treatment. These data should be interpreted cautiously because there are a small number of patients who received many treatments that have large impact on the slope of the linear relationship. Nevertheless, in our clinic the majority of subjects treated with HealthLight™ have significant improvement of their neuropathy symptoms in less than 20 treatments.

The symptoms of diabetic neuropathy are varied. Each alone affects quality of life and is costly to the health care system. This becomes an even more serious problem when several symptoms occur simultaneously. Not surprisingly, patients often seek alternatives to conventional medical treatments for these diabetic neuropathy symptoms.

Our clinics treat a variety of painful conditions. This case report summarizes our results in our subjects who initially presented with painful symptoms related to diabetes. They also scored well below average in a PNQ, a RLSQ and in the TCSS. One might not be impressed by a low score in one of these tests at baseline but all 3 tests indicated the likelihood that this was indeed symptomatic, painful, diabetic neuropathy.

Pain, as well as the troublesome symptoms accompanying restless leg, improved in these subjects. The treatment of the affected limbs took only 20 minutes making the thrice weekly visits to our clinic acceptable by both subjects as well as our clinic staff. Compliance was excellent. There are over 26 million people with diabetes and many, if not most, will develop neuropathy symptoms, specifically pain, that may be lessened by medical care offered by healthcare professionals treating pain, including Podiatrists, Chiropractors and Physical Therapists.

Collectively, an approximately 30-40% improvement in diabetic neuropathy symptoms was seen when this medical device was used on the symptomatic lower extremities. HealthLight™ is cleared to increase circulation. As mentioned above, this could improve nerve function by restoring a presumably reduced blood flow caused by EBG. One mechanism may involve increased release of nitric oxide from hemoglobin. While HealthLight™ was the light therapy device used in this study, other light therapy devices such as InLight or numerous others could be used with the inventive method and would fall within the scope of the present method.

This was an observational study only and any conclusions must be tempered because no subjects were either left untreated or were treated with a placebo device. Nevertheless, in the real world, unlike during clinical trials where there are several exclusion criteria, some subjects complaining of diabetic neuropathy symptoms may be helped by treatments that include light therapy.

With reference to FIG. 19A, Chart 1 depicts Box Plots of Initial Pain, Final Pain and Change from Baseline Pain. Initial pain is skewed high (most subjects had clinically significant pain) but both final pain and change from baseline appear to be nearly symmetric in response to Healthlight™ treatments.

With reference to FIG. 19B, Chart 2 depicts Box Plots of %T-Pre(L), %T-Post (L) and Change from Baseline TCSS Left. The baseline TCSS Left is skewed low, indicating that the majority of subjects had more symptomatic problems but both final and change from baseline following Healthlight™ treatment appear to be nearly symmetric. In other words, despite the severity of symptoms in some subjects on their left leg, improvements after light therapy were similar to those with less severe symptoms.

With reference to FIG. 19C, Chart 3 depicts Box Plots of %T-Pre(R), %T-Post (R) and Change from Baseline TCSS Right. These data indicate that there was more variance in the right leg than the left leg (FIG. 2). NIR treatments improved (% change) by about 25% in both legs.

What is remarkable when reviewing the near infrared light therapy was the fact that the lower the threshold level of the neuropathic symptoms, the better the treatment outcome. In cases of high severity or threshold levels “light” alone was not sufficient over a large number of treatments. However, when the high threshold patients were initially treated repeatedly by acoustic shock wave therapy, the threshold level could be decreased to moderate or even low and then the light therapy could effectively be used in a modest number of treatments of 3 to 5 treatments to achieve a desired effect of reducing the pain and the symptoms as shown in FIGS. 16A-16D using the light device of FIG. 17.

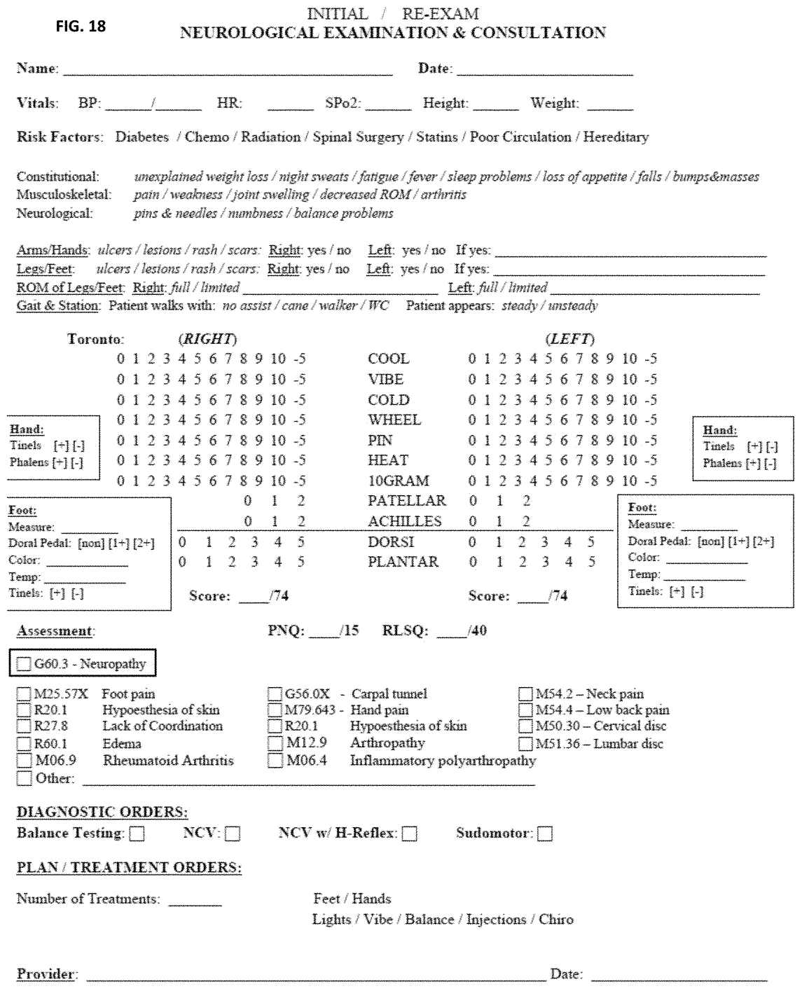

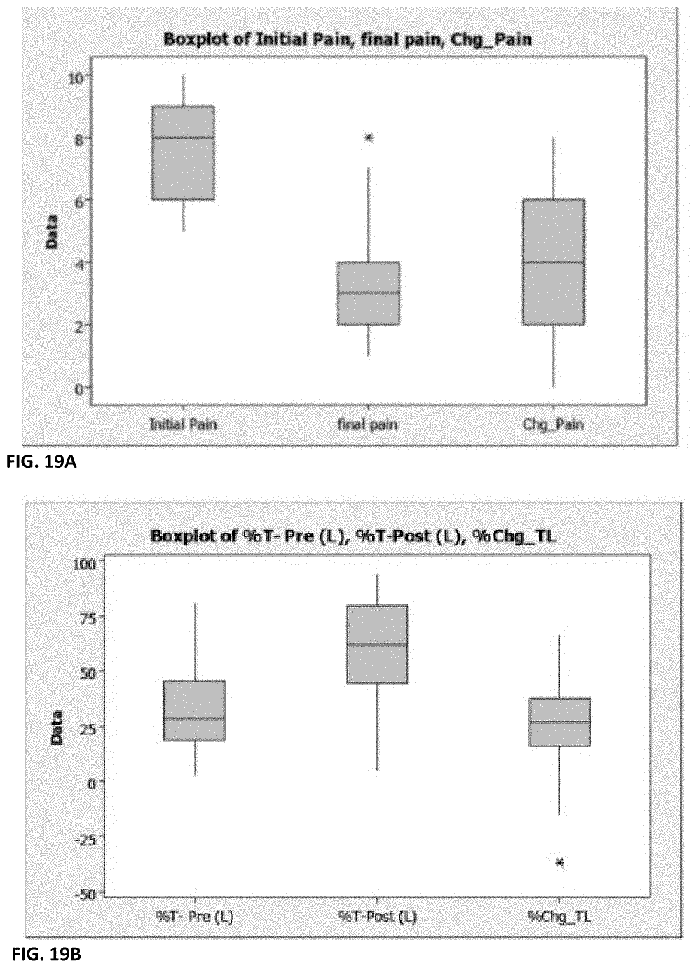

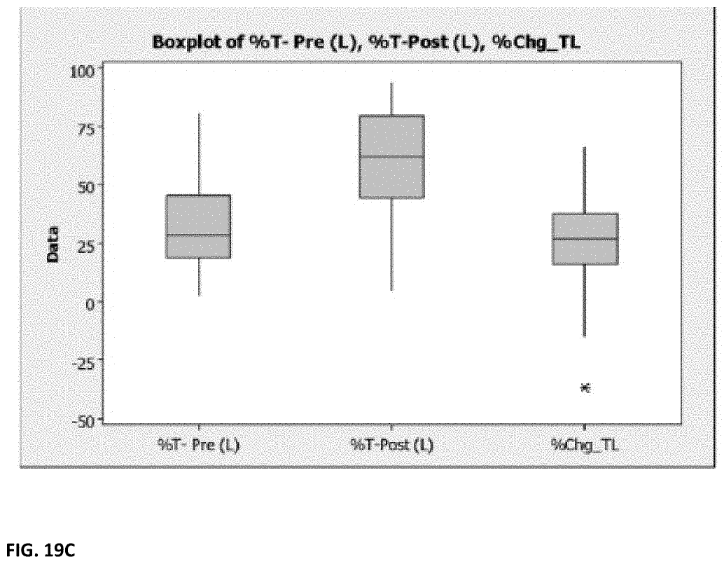

In the present inventive method, one of the steps described is establishing the severity of the neuropathy symptoms and using a threshold level of severity. The inventor used a quantitative score derived from a modified Toronto Clinical Score. An exemplary scoring sheet is shown in FIG. 18. While it is believed the establishing the severity of the neuropathy symptom is important as it dictates the treatment protocol, the threshold level could be based on other ways of establishing the severity. One example may be the judgement and experience of the physician. Another more quantitative method would be to use elevated blood sugar levels as an indicator or any other reasonable qualitative or quantitative measure. Any of which would fall within the scope of the present invention.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient with diabetic neuropathy symptoms comprises the steps of:
establishing the severity of the neuropathy symptoms;
for those patients with symptoms below a threshold level of severity, treating an extremity of those patients with acoustic shock waves followed by near infrared light therapy; and
for patients at or exceeding the threshold level of severity, treating an extremity of those patients with one or more acoustic shock wave treatments over several weeks, monitoring for a reduction in the severity of neuropathy symptoms to below the threshold level of severity and thereafter following an acoustic shock wave treatment, treating the extremity with a near infrared light therapy.

2. The method of claim 1 wherein the step of treating the patient with acoustic shock waves includes the steps of activating an acoustic shock wave generator or source to emit acoustic shock waves through an extremity of the patient; stimulating the sensory nerves of the extremity to rehabilitate and restore function thereby reducing the severity of the diabetic neuropathy symptoms wherein the extremity is positioned in a path of the emitted shock waves.

3. The method of claim 2 wherein the shock wave generator or source is one of ballistic, radial, piezoelectric, or electrohydraulic.

4. The method of claim 3 wherein the emitted shock waves are radial, focused, non-focused, planar, nearly planar, convergent or divergent.

5. The method of claim 4 wherein the acoustic shock waves have a pressure pulse power density in the range of 0.1 to 1.0 mP.

6. The method of claim 1 further comprises the step of measuring the patient's blood glucose level as part of the step of establishing the severity of the neuropathy symptoms.

7. The method of claim 1 wherein the step of establishing the severity of the neuropathy symptoms includes scoring the severity of the neuropathy in an extremity using the Toronto Clinical Scoring System TCSS.

8. The method of claim 1 wherein the patient is diabetic exhibiting type 1 or type 2 diabetes condition.

9. The method of claim 1 wherein the extremity is a leg.

10. The method of claim 1 wherein the extremity is a foot.

11. The method of claim 1 wherein the extremity is an arm.

12. The method of claim 1 wherein the patient has an elevated baseline blood sugar level prior to treating which lowers after treatment.

13. The method of claim 1 wherein repeating the method periodically a plurality of times over a period of weeks to lower said baseline level of blood sugar.

14. The method of claim 1 comprises the steps of: identifying a diabetic at risk patient of neuropathy symptoms, the patient having an at risk baseline blood sugar level; and
subjecting the at risk extremity to shock waves to lower said baseline sugar level.

15. The method of claim 14 wherein the step of identifying an at risk patient includes one or more indications of risk based on family history, genetic disposition, physical condition, or blood or extremity analysis.

16. The method of claim 14 further comprises the step of testing the at risk extremity to establish measured the baseline condition pre shock wave therapy.

17. The method of claim 14 further comprises the step of post shockwave therapy testing the blood sugar level for comparison to the baseline condition.

18. The method of claim 1 wherein each treated extremity is exposed to a treatment of greater than 500 shock waves.

19. The method of claim 18 wherein each treated extremity is exposed to a treatment of less than 2000 shock waves.

20. The method of claim 18 wherein each extremity is exposed to a treatment of about 600 shock waves.

21. The method of claim 1 wherein each extremity is exposed to a near infrared light over a treatment duration of greater than 10 minutes.

22. The method of claim 21 wherein the treatment is about 20 minutes.

23. The method of claim 1 wherein the diabetic neuropathy symptoms of Chemotherapy Induced Peripheral Neuropathy (CIPN) are of sufficient severity to stop chemotherapy treatments.

24. The method of claim 23 further comprises the steps of reducing the symptoms and reinitiating chemotherapy treatments.

25. A method of pre-treating a patient prior to or during chemotherapy with diabetic neuropathy symptoms or at risk of having chemotherapy induced peripheral neuropathy comprises the steps of:
establishing the severity of the neuropathy symptoms;
for those patients with symptoms below a threshold level of severity, treating an extremity of those patients with acoustic shock waves followed by near infrared light therapy;
for patients at or exceeding the threshold level of severity, treating an extremity of those patients with one or more acoustic shock wave treatments over several weeks, monitoring for a reduction in the severity of neuropathy symptoms to below the threshold level of severity and thereafter following an acoustic shock wave treatment, treating the extremity with a near infrared light therapy;
treating the patient with chemotherapy; and
monitoring the neuropathy symptoms if any periodically during the duration of chemotherapy.

26. The method of claim 25 wherein the step of establishing the severity further includes establishing a baseline using the Toronto Clinical Score prior to the patient starting chemotherapy.

27. The method of claim 25 wherein the step of monitoring the neuropathy symptoms is delayed 24 hours post chemotherapy infusion to allow the majority of chemo to dissipate out of the patient's system.
